(19) United States
(12) Patent Application Publication
(10) Pub. No.: US 2026/0186181 A1
(43) Pub. Date: Jul. 2, 2026

(54) DIFFRACTIVE OPTICAL ELEMENT, METHOD FOR MANUFACTURING SAME, HOLOGRAM USING SAME, VR/AR DEVICE USING SAME, AND HUD USING SAME

(71) Applicant: KOREA UNIVERSITY RESEARCH AND BUSINESS FOUNDATION, Seoul (KR)

(72) Inventors: Seungwoo LEE, Seoul (KR); Yongjun LIM, Pohang-si (KR)

(73) Assignee: KOREA UNIVERSITY RESEARCH AND BUSINESS FOUNDATION, Seoul (KR)

(21) Appl. No.: 19/549,752

(22) Filed: Feb. 25, 2026

Related U.S. Application Data
(63) Continuation of application No. PCT/KR2024/012014, filed on Aug. 12, 2024.

(30) Foreign Application Priority Data
Aug. 31, 2023 (KR) 10-2023-0115176
Feb. 19, 2024 (KR) 10-2024-0023647

(51) Int. Cl. G02B 5/18

(52) U.S. Cl. CPC G02B 5/1852

(57) ABSTRACT

Provided is a method for manufacturing a diffractive optical element. The method for manufacturing a diffractive optical element may comprise: a step of preparing an imprinting stamp including a stamp pattern in which peaks and valleys having a curved shape are alternately and repeatedly arranged; a step of preparing a base structure in which a coating layer including a photocurable polymer and nanoparticles is formed on a base substrate; and a step of applying pressure to the coating layer of the base structure using the imprinting stamp to form, in the coating layer, a base pattern having the inverse image of the stamp pattern.

### CROSS-REFERENCE TO RELATED APPLICATIONS

The present application is a continuation of International Patent Application No. PCT/KR2024/012014, filed Aug. 12, 2024, which is based upon and claims the benefit of priority to Korean Patent Application Nos. 10-2023-0115176, filed on Aug. 31, 2023 and 10-2024-0023647 filed Feb. 19, 2024. The disclosures of the above-listed applications are hereby incorporated by reference herein in their entirety.

### TECHNICAL FIELD

The present invention relates to a diffractive optical element and a manufacturing method thereof, and more particularly, to a diffractive optical element and a manufacturing method thereof having a pattern formed on a transparent material with high refractive index.

The diffractive optical element and the manufacturing method thereof according to the embodiments of the present invention may be used in a hologram, a virtual reality (VR)/augmented reality (AR) device, and a head up display (HUD).

### BACKGROUND ART

A diffractive optical element refers to an optical element used in various optical fields from spectrometers and analog holograms to digital holograms.

Recently, a method for manufacturing an optical element by using polymers containing azobenzene molecules has been extensively studied. This technology enables forming a structure simply by irradiating a specific light pattern without a chemical etching process so as to facilitate a manufacturing process and improve yields.

In addition, the above optical element, unlike diffractive optical elements that may be manufactured using other manufacturing methods, has a surface structure with a sinusoidal wave shape so that optical loss can be reduced.

However, this optical element may not be used in the entire visible light range due to the absorption characteristics of the material, and may also be limited in diffraction efficiency, thus there are limits in application.

Thus, the present invention provides a diffractive optical element and a manufacturing method thereof in which the diffractive optical element is manufactured using a polymer containing azobenzene molecules, so as to be easily used in the entire visible light range and implement high diffraction efficiency.

### DISCLOSURE

### Technical Problem

One technical problem to be solved by the present invention is to provide a diffractive optical element and a manufacturing method thereof in which polymer containing azobenzene molecules is used.

Another technical problem to be solved by the present invention is to provide a diffractive optical element and a manufacturing method thereof in which an absorption rate is reduced.

Still another technical problem to be solved by the present invention is to provide a diffractive optical element and a manufacturing method thereof in which transmittance is improved.

Still another technical problem to be solved by the present invention is to provide a diffractive optical element and a manufacturing method thereof in which refractive index is improved.

Still another technical problem to be solved by the present invention is to provide a diffractive optical element and a manufacturing method thereof in which a diffraction efficiency is improved.

Still another technical problem to be solved by the present invention is to provide a diffractive optical element and a manufacturing method thereof in which a manufacturing process is simplified and a process cost is reduced.

The technical problems to be solved by the present invention are not limited to the above-mentioned technical problems.

### Technical Solution

In order to solve the above-mentioned technical problems, the present invention provides a method for manufacturing a diffractive optical element.

According to the one embodiment, the method for manufacturing the diffractive optical element includes: a step of preparing an imprinting stamp including a stamp pattern in which peaks and valleys having a curved shape are alternately and repeatedly arranged in at least some areas; a step of preparing a base structure in which a coating layer including a photocurable polymer and nanoparticles is formed on a base substrate; and a step of applying pressure to the coating layer of the base structure using the imprinting stamp to form, in the coating layer, a base pattern having the reverse phase of the stamp pattern.

According to the one embodiment, the step of preparing the imprinting stamp may include: a step of preparing a sub-structure in which a polymer film containing azobenzene molecules is formed on a sub-substrate; a step of irradiating light onto the polymer film to form a polymer pattern in which peaks and valleys having a curved shape are alternately and repeatedly arranged in at least some areas; a step of coating a stamp composition including a thermosetting polymer on the polymer film to cover the polymer pattern; and a step of manufacturing the imprinting stamp including the stamp pattern having a reverse phase of the polymer pattern by heat-treating the stamp composition.

According to the one embodiment, the base pattern may have peaks and valleys having a curved shape alternately and repeatedly arranged, in which the peak of the base pattern may correspond to the valley of the stamp pattern, and the valley of the base pattern may correspond to the peak of the stamp pattern.

According to the one embodiment, the step of preparing the base structure may include: a step of preparing a base substrate; a step of forming an adhesive layer on the base substrate; and a step of forming the coating layer on the adhesive layer.

According to the one embodiment, the adhesive layer may include poly(methyl methacrylate) (PMMA).

According to the one embodiment, the method for manufacturing the diffractive optical element may further include: a step of treating the base substrate by using oxygen plasma, after the preparing of the base substrate and before the forming of the adhesive layer.

According to the one embodiment, the method for manufacturing the diffractive optical element may further include: a step of imparting hydrophobicity to a surface of the imprinting stamp on which the stamp pattern is formed, after the preparing of the imprinting stamp and before the forming of the base pattern.

According to the one embodiment, the surface of the imprinting stamp on which the stamp pattern is formed may be treated with hexamethyldisilazane (HMDS) to impart the hydrophobicity.

According to the one embodiment, the surface of the imprinting stamp on which the stamp pattern is formed may be further disposed thereon with a hydrophobicity-imparted layer, and the base structure may further include an adhesive layer disposed between the base substrate and the coating layer, such that a ΔW value derived through <Equation 1> below may be 40 mJ/m² or more.

$$\\ \begin{matrix}△ W = \vert 4 ( \frac{{γ}_{s 1}^{d} {γ}_{s 2}^{d}}{{γ}_{s 1}^{d} + {γ}_{s 2}^{d}} + \frac{{γ}_{s 1}^{p} {γ}_{s 2}^{p}}{{γ}_{s 1}^{p} + {γ}_{s 2}^{p}} ) - 4 ( \frac{{γ}_{s 2}^{d} {γ}_{s 3}^{d}}{{γ}_{s 2}^{d} + {γ}_{s 3}^{d}} + \frac{{γ}_{s 2}^{p} {γ}_{s 3}^{p}}{{γ}_{s 2}^{p} + {γ}_{s 3}^{p}} ) \vert & < Equation 1 >\end{matrix}$$

(γ^{d}: dispersion surface energy, γ^{p}: polar surface energy, s₁: the base substrate, s₂: the coating layer, and s₃: the imprinting stamp)

In order to solve the above technical problem, the present invention provides a diffractive optical element.

According to the one embodiment, in a diffractive optical element including a diffractive optical layer including a polymer and nanoparticles, the diffractive optical layer may include a base pattern in which peaks and valleys having a curved shape are alternately and repeatedly arranged, and a diffraction efficiency may be 30% or more.

According to the one embodiment, the diffractive optical element may have transmittance of 80% or more for light having a wavelength of 400 nm to 800 nm.

According to the one embodiment, the polymer may include dipentaerythritol penta-/hexa-acrylate or benzyl methacrylate (BzMA).

According to the one embodiment, the nanoparticles may include titanium oxide (TiO₂) nanoparticles.

### Advantageous Effects

The diffractive optical element according to the embodiment of the present invention may have a structure in which a sinusoidal wave-shaped pattern is formed on a coating layer having a high refractive index and transparent characteristics, and a polymer containing azobenzene molecules may be used to form the sinusoidal wave-shaped patterns. Accordingly, the sinusoidal wave-shaped pattern can be formed without a complex etching process, so that the process can be simplified, and process costs can be reduced.

In addition, due to the above-described structure (the structure in which the sinusoidal wave-shaped pattern is formed on the coating layer having high refractive index and transparent properties), a high transmittance of 80% or more can be implemented for the entire visible light range (for example, wavelength from 400 nm to 800 nm), so that the diffractive optical element can be easily used over the entire visible light range, and the diffraction efficiency over the entire visible light range can also theoretically have a maximum value.

### DESCRIPTION OF DRAWINGS

FIG. 1 is a flowchart for explaining a method for manufacturing a diffractive optical element according to the embodiment of the present invention.

FIG. 2 is a flowchart for specifically explaining step S100 of the method for manufacturing the diffractive optical element according to the embodiment of the present invention.

FIG. 3 is a schematic diagram for explaining step S110 of the method for manufacturing the diffractive optical element according to the embodiment of the present invention.

FIG. 4 is a schematic diagram for explaining step S120 of the method for manufacturing the diffractive optical element according to the embodiment of the present invention.

FIG. 5 is a schematic diagram for explaining step S130 of the method for manufacturing the diffractive optical element according to the embodiment of the present invention.

FIG. 6 is a schematic diagram for explaining step S140 of the method for manufacturing the diffractive optical element according to the embodiment of the present invention.

FIG. 7 is a schematic diagram for explaining step S150 of the method for manufacturing the diffractive optical element according to the embodiment of the present invention.

FIG. 8 is a flowchart for specifically explaining step S200 of the method for manufacturing the diffractive optical element according to the embodiment of the present invention.

FIG. 9 is a schematic diagram for explaining steps S210 and S220 of the method for manufacturing the diffractive optical element according to the embodiment of the present invention.

FIG. 10 is a schematic diagram for explaining steps S230 and S240 of the method for manufacturing the diffractive optical element according to the embodiment of the present invention.

FIGS. 11 and 12 are schematic diagrams for explaining step S300 of the method for manufacturing the diffractive optical element according to the embodiment of the present invention.

FIGS. 13A and 13B are photographs of a coating layer of a diffractive optical element according to an experimental example of the present invention.

FIG. 14 is a view for explaining results of measuring transmittances for an adhesive layer and a coating layer in the diffractive optical element according to the experimental example of the present invention.

FIG. 15 is a view for explaining results of measuring refractive index of a coating layer in the diffractive optical element according to the experimental example of the present invention.

FIG. 16 is a view for explaining a theoretical diffraction efficiency of the diffractive optical element according to the experimental example of the present invention.

FIG. 17 is a view comparing an actual diffraction efficiency with a theoretical diffraction efficiency of the diffractive optical element according to the experimental example of the present invention.

FIGS. 18A and 18B are views comparing diffraction tendencies between the diffractive optical element according to the experimental example of the present invention and a diffractive optical element according to a comparative example.

FIG. 19A to FIG. 20D are views for explaining influences of bonding work values in a manufacturing process of the diffractive optical element according to the experimental example of the present invention.

FIG. 21A to FIG. 22D are views for explaining influences of photocurable polymer and a nanoparticle content in the a manufacturing process of the diffractive optical element according to the experimental example of the present invention.

### BEST MODE

### Mode for Invention

Hereinafter, exemplary embodiments of the present invention will be described in detail with reference to the accompanying drawings. However, the technical idea of the present invention is not limited to the exemplary embodiments described herein and may be embodied in other forms. Further, the embodiments are provided to enable contents disclosed herein to be thorough and complete and provided to enable those skilled in the art to fully understand the idea of the present invention.

In the specification, when one component is mentioned as being on another component, it signifies that the one component may be placed directly on another component or a third component may be interposed therebetween. In addition, in drawings, thicknesses of films and regions may be exaggerated to effectively describe the technology of the present invention.

In addition, although terms such as first, second and third are used herein to describe various components in various embodiments of the present specification, the components will not be limited by the terms. The above terms are used merely to distinguish one component from another. Accordingly, a first component referred to in one embodiment may be referred to as a second component in another embodiment. Each embodiment described and illustrated herein may also include a complementary embodiment. In addition, the term “and/or” is used herein to include at least one of the components listed before and after the term.

The singular expression herein includes a plural expression unless the context clearly specifies otherwise. In addition, it will be understood that the term such as “include” or “have” herein is intended to designate the presence of feature, number, step, component, or a combination thereof recited in the specification, and does not preclude the possibility of the presence or addition of one or more other features, numbers, steps, components, or combinations thereof. In addition, the term “connection” is used herein to include both indirectly connecting a plurality of components and directly connecting the components.

In addition, in the following description of the embodiments of the present invention, the detailed description of known functions and configurations incorporated herein will be omitted when it possibly makes the subject matter of the present invention unclear unnecessarily.

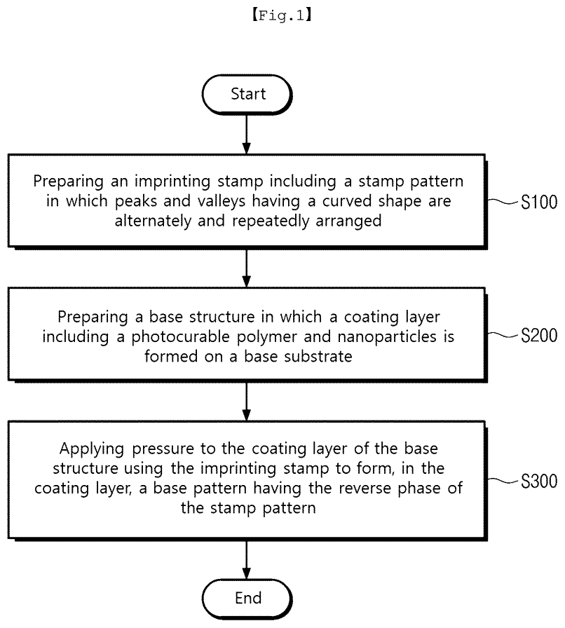

FIG. 1 is a flowchart for explaining a method for manufacturing a diffractive optical element according to the embodiment of the present invention.

Referring to FIG. 1, the method for manufacturing the diffractive optical element according to the embodiment of the present invention may include a step of preparing an imprinting stamp (S100), a step of preparing a base structure on which a coating layer is formed (S200), and a step of applying pressure to the coating layer of the base structure by using the imprinting stamp to form a base pattern on the coating layer (S300). Hereinafter, each step will be described.

### Step of Preparing an Imprinting Stamp (S100)

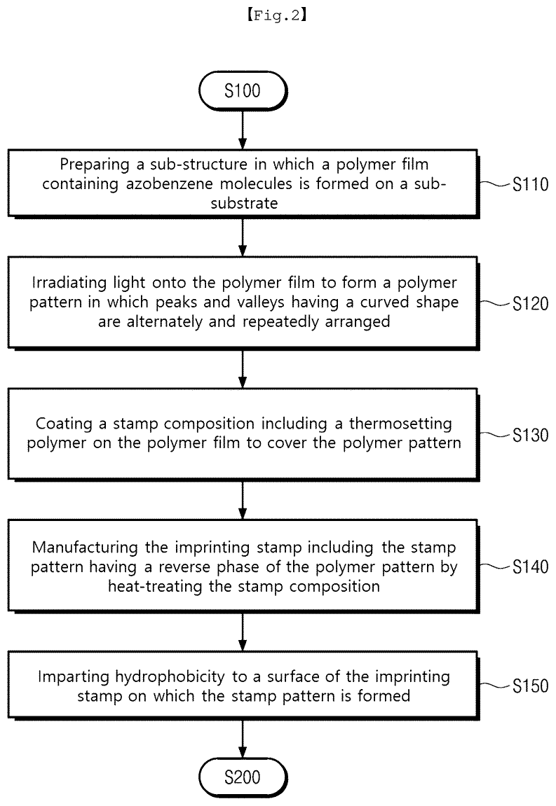

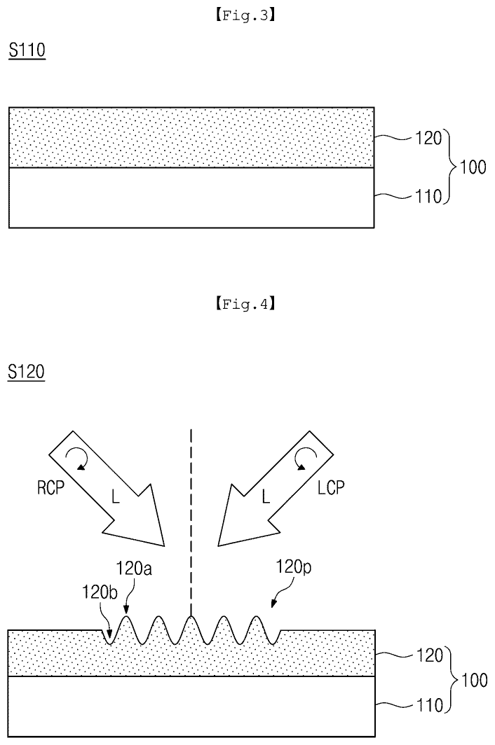

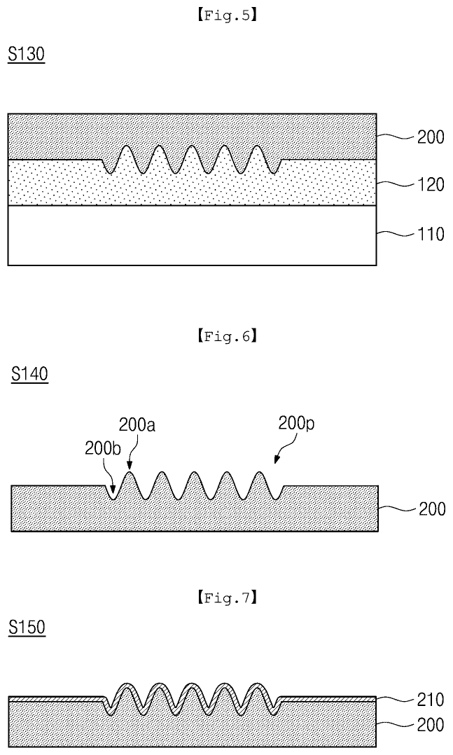

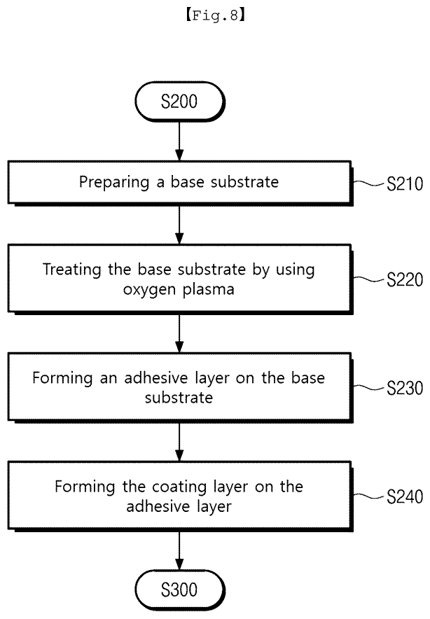

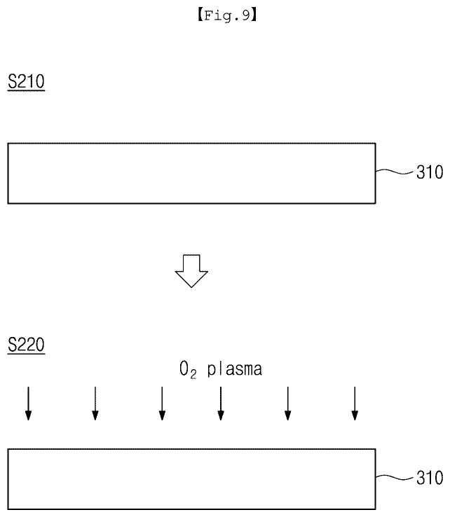

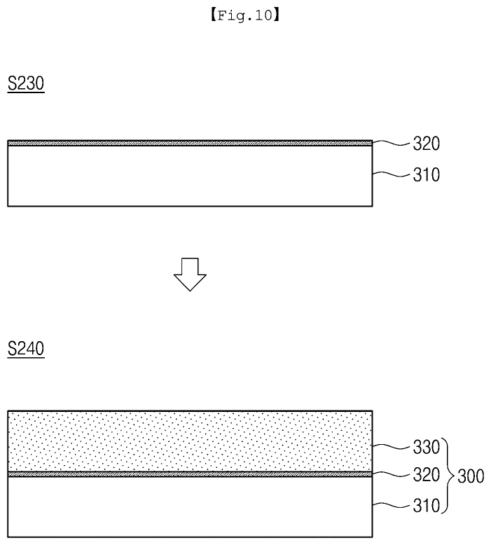

FIG. 2 is a flowchart for specifically explaining step S100 of the method for manufacturing the diffractive optical element according to the embodiment of the present invention; FIG. 3 is a schematic diagram for explaining step S110 of the method for manufacturing the diffractive optical element according to the embodiment of the present invention; FIG. 4 is a schematic diagram for explaining step S120 of the method for manufacturing the diffractive optical element according to the embodiment of the present invention; FIG. 5 is a schematic diagram for explaining step S130 of the method for manufacturing the diffractive optical element according to the embodiment of the present invention; FIG. 6 is a schematic diagram S140 of the method for manufacturing the diffractive optical element according to the embodiment of the present invention; and FIG. 7 is a schematic diagram for explaining step S150 of the method for manufacturing the diffractive optical element according to the embodiment of the present invention.

Referring to FIGS. 2 and 3, a sub-structure 100 in which a polymer film 120 containing azobenzene molecules is formed on a sub-substrate 110 may be prepared (S110). According to the one embodiment, the sub-substrate 110 may include a glass substrate. According to the one embodiment, the polymer film 120 may include poly(disperse red 1 methacrylate) (pDR1m).

Referring to FIGS. 2 and 4, light L may be irradiated onto the polymer film 120, so as to form a polymer pattern 120p in which peaks 120a and valleys 120b having a curved shape are alternately and repeatedly arranged in at least some areas (S120).

According to the one embodiment, the light L irradiated onto the polymer film 120 may include a polarization interference pattern (PIP). More particularly, the light L irradiated onto the polymer film 120 may include a polarization interference pattern in which right circularly polarized (RCP) light and left circularly polarized (LCP) light are mixed.

More particularly, when the light L including the polarization interference pattern is irradiated onto the polymer film 120, azobenzene molecules may be aligned perpendicular to the polarization direction and move in the polarization direction. Accordingly, the polymer pattern 120p in which peaks 120a and valleys 120b having a curved shape are alternately and repeatedly arranged in at least some areas on the polymer film 120. In other words, due to the properties of azobenzene molecules, the simple method of irradiating light without an etching process is available, and accordingly, a sinusoidal wave-shaped pattern may be formed on the polymer film 120.

Referring to FIGS. 2 and 5, a stamp composition 200 may be coated on the polymer film 120 to cover the polymer pattern 120p (S130). According to the one embodiment, the stamp composition 200 may include a thermosetting polymer. For example, the thermosetting polymer may include polydimethylsiloxane (PDMS).

Referring to FIGS. 2 and 6, the stamp composition 200 may be heat-treated to manufacture an imprinting stamp 200 including a stamp pattern 200p having a reverse phase of the polymer pattern 120p (S140). In other words, the imprinting stamp 200 may be defined as a state in which the stamp composition 200 is cured. In addition, the imprinting stamp 200 may be prepared in a state separated from the sub-structure 100.

As described above since the stamp pattern 200p of the imprinting stamp 200 has the reverse phase of the polymer pattern 120p, the stamp pattern 200p may also have a structure in which peaks 200a and valleys 200b are alternately and repeatedly arranged. The peak 200a of the stamp pattern 200p may correspond to the valley 120b of the polymer pattern 120p, and the valley 200b of the stamp pattern 200p may correspond to the peak 120a of the polymer pattern 120p.

Referring to FIGS. 2 and 7, hydrophobicity may be imparted to a surface of the imprinting stamp 200 on which the stamp pattern 200p is formed (S150). More particularly, the surface of the imprinting stamp 200 on which the stamp pattern 200p is formed may be treated with hexamethyldisilazane (HMDS) to impart the hydrophobicity. Accordingly, a hydrophobicity-imparted layer 210 may be formed on the surface of the imprinting stamp 200 on which the stamp pattern 200p is formed.

A separation between a base structure 300 and the imprinting stamp 200 may be easily achieved in step S300 described later by the hydrophobicity-imparted layer 210. In contrast, when the hydrophobicity-imparted layer 210 is absent, a problem may occur in which the separation between the base structure 300 and the imprinting stamp 200 is not properly achieved in S300 step described later.

### Step of Preparing a Base Structure (S200)

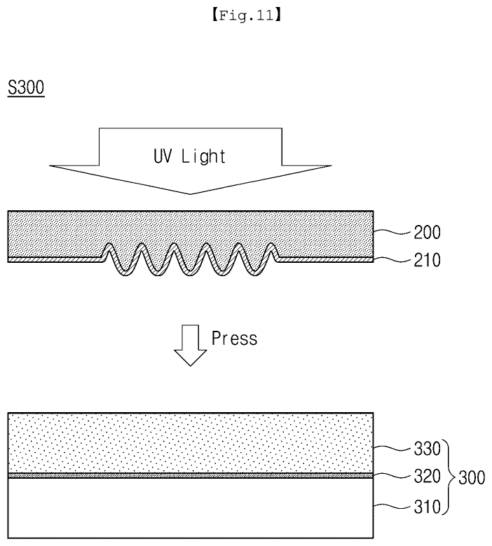

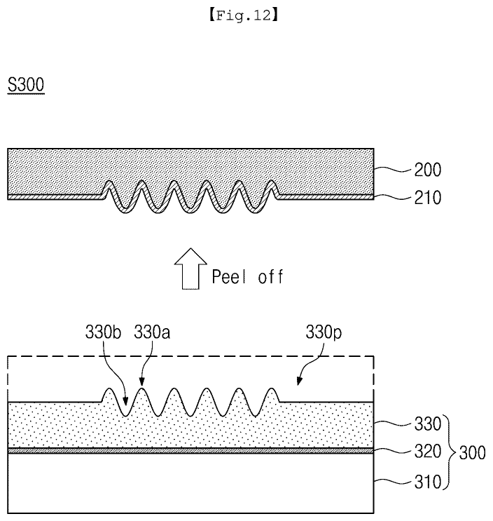

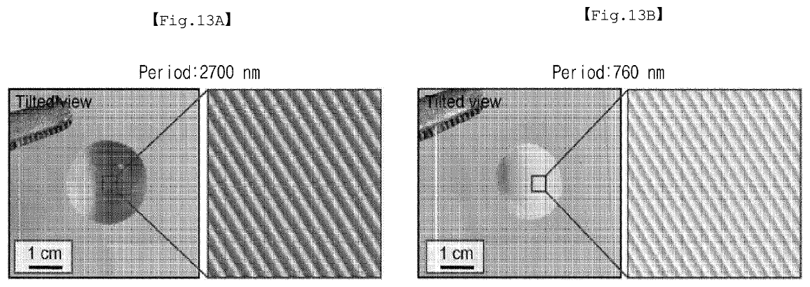

FIG. 8 is a flowchart for specifically explaining step S200 of the method for manufacturing the diffractive optical element according to the embodiment of the present invention; FIG. 9 is a schematic diagram for explaining steps S210 and S220 of the method for manufacturing the diffractive optical element according to the embodiment of the present invention; and FIG. 10 is a schematic diagram for explaining steps S230 and S240 of the method for manufacturing the diffractive optical element according to the embodiment of the present invention.

Referring to FIGS. 8 and 9, a base substrate 310 may be prepared (S210). According to the one embodiment, the above base substrate 310 may include a glass substrate.

The base substrate 310 may be treated with oxygen plasma (O₂ plasma) (S220). Accordingly, adhesion between the adhesive layer described below and the base substrate 310 may be further improved. In contrast, when the oxygen plasma treatment step is omitted, a problem may occur in which the adhesive layer described later is not easily formed.

Referring to FIGS. 8 and 10, an adhesive layer 320 may be formed on the base substrate 310 (S230). According to the one embodiment, the adhesive layer 320 may include poly(methyl methacrylate) (PMMA). The adhesive layer 320 may improve adhesion between the coating layer described later and the base substrate 310.

A coating layer 330 may be formed on the adhesive layer 320 (S240). Thus, a base structure 300 may be prepared. According to the one embodiment, the coating layer 330 may be formed through coating of a base composition having a high refractive index and transparent properties. Accordingly, the coating layer 330 may also have the high refractive index and the transparent properties.

According to the one embodiment, the base composition may include a photocurable polymer, nanoparticles, a photoinitiator, and a solvent. For example, the photocurable polymer may include dipentaerythritol penta-/hexa-acrylate or benzyl methacrylate (BzMA). For example, the nanoparticles may include titanium oxide (TiO₂) nanoparticles as metal nanoparticles capable of improving the refractive index. For example, the photoinitiator may include Igacure 784 and may be used in an amount of 1 wt % based on a total weight of the base composition. For example, the solvent may include propylene glycol monomethyl ether acetate (PGMEA).

According to the one embodiment, reliability of the diffractive optical layer described later may be controlled according to the content of the photocurable polymer and the nanoparticles in the base composition.

For example, when the photocurable polymer includes dipentaerythritol penta-/hexa-acrylate and the nanoparticles include titanium oxide (TiO₂) nanoparticles, the content of the nanoparticles: the photocurable polymer may be controlled to be greater than 70:30 wt % and less than 95:5 wt %. Accordingly, the reliability of the diffractive optical layer described later may be improved. In contrast, when the content of the nanoparticles: the photocurable polymer is controlled to 70:30 wt % or less, the reliability may be lowered due to aggregation. In addition, when the content of the nanoparticles: the photocurable polymer is controlled to 95:5 wt % or more, the reliability may be lowered due to cracks.

For another example, when the photocurable polymer includes benzyl methacrylate (BzMA) and the nanoparticles include titanium oxide (TiO₂) nanoparticles, the content of the nanoparticles: the photocurable polymer may be controlled to be less than 95:5 wt %. Accordingly, the reliability of the diffractive optical layer described later may be improved. In contrast, when the content of the nanoparticles: the photocurable polymer is controlled to 95:5 wt % or more, the reliability may be lowered due to cracks.

### Step of Forming a Base Pattern (S300)

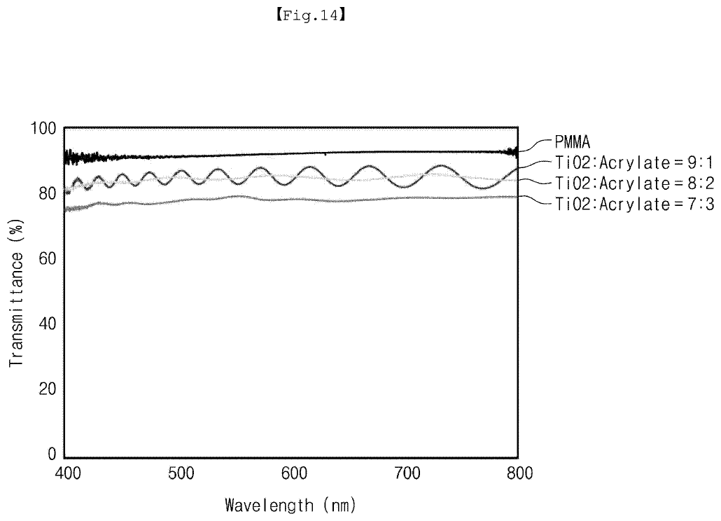

FIGS. 11 and 12 are schematic diagrams for explaining step S300 of the method for manufacturing the diffractive optical element according to the embodiment of the present invention.

Referring to FIGS. 1, 11 and 12, after the coating layer 330 is irradiated with ultraviolet (UV) light while applying pressure to the coating layer 330 of the base structure 300 using the imprinting stamp 200, the imprinting stamp 200 may be peeled off from the base structure 300. Accordingly, a base pattern 330p having a reverse phase of the stamp pattern 200p may be formed on the coating layer 330 (S300).

More particularly, the pressure may be applied and the ultraviolet light may be irradiated while the surface of the imprinting stamp 200 on which the hydrophobicity-imparted layer 210 is formed comes into contact with the coating layer 330. Accordingly, the base pattern 330p having the reverse phase of the stamp pattern 200p may be formed on the coating layer 330.

Since the base pattern 330p has the reverse phase of the stamp pattern 200p, the base pattern 330p may also have a structure in which peaks 330a and valleys 330b are alternately and repeatedly arranged. The peak 330a of the base pattern 330p may correspond to the valley 200b of the stamp pattern 200p, and the valley 330b of the base pattern 300p may correspond to the peak 200a of the stamp pattern 200p. In other words, a sinusoidal wave-shaped pattern may be formed on the coating layer 330 having the high refractive index and the transparent properties.

It may be difficult to form a sinusoidal wave-shaped pattern in a polymer material having a high refractive index without an etching process. Accordingly, in order to form a sinusoidal wave-shaped pattern in a polymer material having a high refractive index without an etching process, the present invention provides a method in which a sub-structure is formed using an azobenzene-based polymer having a low refractive index but capable of easily forming the sinusoidal wave-shaped pattern without an etching process, an imprinting stamp is manufactured using the sub-structure and then a sinusoidal wave-shaped pattern is formed on a polymer material having a high refractive index through the imprinting stamp.

According to the one embodiment, in the process of separating the imprinting stamp 200 from the base structure 300, a problem may occur in which the coating layer 330 fails to remain on the adhesive layer 320 and is separated from the adhesive layer 320 together with the imprinting stamp 200. Accordingly, in order to solve the above-mentioned problem, a value in work of adhesion between the base structure 300 and the imprinting stamp 200 may be controlled. More particularly, a ΔW value derived through <Equation 1> below may be controlled to be 40 mJ/m² or more.

$$\\ \begin{matrix}△ W = \vert 4 ( \frac{{γ}_{s 1}^{d} {γ}_{s 2}^{d}}{{γ}_{s 1}^{d} + {γ}_{s 2}^{d}} + \frac{{γ}_{s 1}^{p} {γ}_{s 2}^{p}}{{γ}_{s 1}^{p} + {γ}_{s 2}^{p}} ) - 4 ( \frac{{γ}_{s 2}^{d} {γ}_{s 3}^{d}}{{γ}_{s 2}^{d} + {γ}_{s 3}^{d}} + \frac{{γ}_{s 2}^{p} {γ}_{s 3}^{p}}{{γ}_{s 2}^{p} + {γ}_{s 3}^{p}} ) \vert & < Equation 1 >\end{matrix}$$

(γ^{d}: dispersion surface energy, γ^{p}: polar surface energy, s₁: the base substrate, s₂: the coating layer, and s₃: the imprinting stamp)

The coating layer 330 on which the base pattern 330p is formed may be defined as a diffractive optical layer, and an element including the diffractive optical layer may be defined as a diffractive optical element. The diffractive optical layer has the structure in which the sinusoidal wave-shaped base pattern 330p is formed on the coating layer 330 having the high refractive index and the transparent characteristics, so that the diffraction efficiency over the entire visible light range may theoretically have a maximum value. More particularly, the diffractive optical element including the diffractive optical layer may have a diffraction efficiency value of 30% or more derived through <Equation 2> below. In other words, the diffractive optical element including the diffractive optical layer may be calculated using a diffraction efficiency calculation scheme of a thin sinusoidal phase grating.

$$\\ \begin{matrix}{DE}_{m} = {\vert \sum {J}_{m} \frac{{k}_{o} ( {n}^{′} - n ) {h}_{0}}{2} \vert}^{2} & < Equation 2 >\end{matrix}$$

DEₘ: diffraction efficiency, m: diffraction order, k₀: frequency, n′: refractive index of diffractive optical layer, n: refractive index of air (=1), h₀: maximum value of modulation height, and J: Bessel function)

The Bessel function of <Equation 2> may be expressed as <Equation 3> below.

$$\\ \begin{matrix}{J}_{m} ( x ) = {{\sum }^{∞}}_{p = 0} \frac{{( - 1 )}^{p}}{p ! T ( p + m + 1 )} {( \frac{x}{2} )}^{2 p + m} & < Equation 3 >\end{matrix}$$

The conventional diffractive optical element manufactured by the method of forming a sinusoidal wave-shaped pattern by irradiating light on a polymer film containing azobenzene molecules cannot be used in the entire visible light range due to the characteristics of azobenzene molecules (high absorption rate), and may also have a low diffraction efficiency.

However, the diffractive optical element according to the embodiment of the present invention, has the structure in which the sinusoidal wave-shaped base pattern 330p is formed on the coating layer 330 having the high refractive index and the transparent characteristics, and accordingly a high transmittance of 80% or more can be implemented for the entire visible light range (for example, wavelength from 400 nm to 800 nm), so that the diffractive optical element can be easily used over the entire visible light range, and the diffraction efficiency over the entire visible light range can also theoretically have a maximum value. Due to these features, the diffractive optical element according to the embodiment of the present invention may be used in a hologram, a virtual reality (VR)/augmented reality (AR) device, and a head up display (HUD).
the diffractive optical element and the manufacturing method thereof according to the embodiments of the present invention have been described. Hereinafter, results on specific experimental examples and characteristic evaluations of the diffractive optical element and the manufacturing method thereof according to the embodiments of the present invention will be described.

### Experimental Example 1: Manufacturing of Diffractive Optical Element

A glass substrate is coated with a poly(disperse red 1 methacrylate) (pDR1m) polymer film, and then irradiated with light containing a polarization interference pattern to form a polymer pattern having a sinusoidal wave shape. Thereafter, the polymer pattern is coated and covered with polydimethylsiloxane (PDMS) and heat-treated to harden the PDMS, and the hardened PDMS is separated as a polymer film, thereby manufacturing an imprinting stamp in which a sinusoidal wave-shaped stamp pattern is formed on a surface of the hardened PDMS. In addition, the surface of the imprinting stamp on which the stamp is formed pattern is treated with hexamethyldisilazane (HMDS) to impart hydrophobicity.

The glass substrate is treated with oxygen plasma and then poly(methyl methacrylate) (PMMA) is coated on the glass substrate to form an adhesive layer. Thereafter, the adhesive layer is coated with a base composition including a photocurable polymer, nanoparticles, a photoinitiator, and a solvent to form a coating layer, thereby manufacturing a base structure. More particularly, dipentaerythritol penta-/hexa-acrylate or benzyl methacrylate (BzMA) is used as the photocurable polymer, titanium oxide (TiO₂) nanoparticles are used as the nanoparticles, Igacure 784 (1 wt % based on the total weight of the base composition) is used as the photoinitiator, and propylene glycol monomethyl ether acetate (PGMEA) is used as the solvent.

Finally, pressure is applied to the base structure with the imprinting stamp while the coating layer of the base structure coming into contact with the stamp pattern of the imprinting stamp, and the coating layer is irradiated with UV light to cure the coating layer. Thereafter, the base structure is peeled off from the imprinting stamp. Accordingly, a diffractive optical element is manufactured in which a sinusoidal wave-shape pattern is formed on the coating layer.

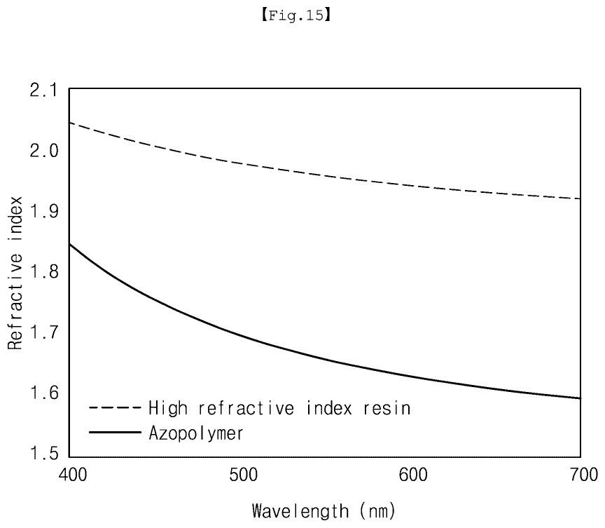

FIGS. 13A and 13B are photographs of a coating layer of a diffractive optical element according to an experimental example of the present invention.

FIG. 13A shows a photographed state in which the base pattern of the coating layer is formed with a period of 2700 nm, and FIG. 13B shows a photographed state in which the base pattern of the coating layer is formed with a period of 760 nm. As can be seen in FIGS. 13A and 13B, the base pattern of the diffractive optical element according to the Experimental Example has a highly reliable sinusoidal wave shape.

### Experimental Example 2: Measurement on Transmittance and Refractive Index of Diffractive Optical Element

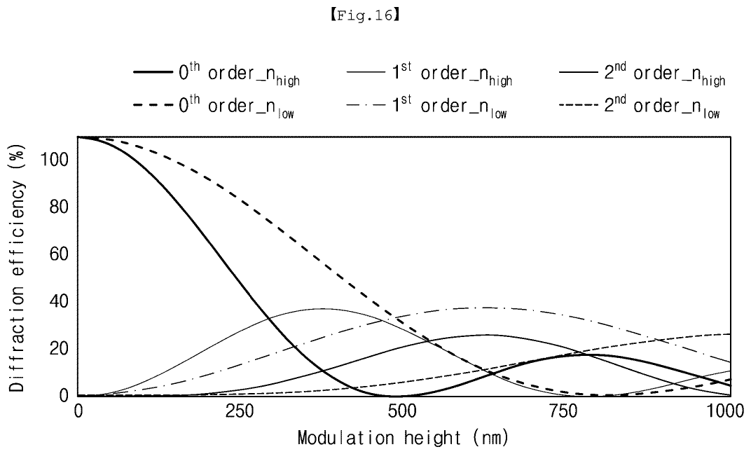

FIG. 14 is a view for explaining results of measuring transmittances for an adhesive layer and a coating layer in the diffractive optical element according to the experimental example of the present invention.

Referring to FIG. 14, transmittance is measured for each of the adhesive layer (PMMA) and the coating layer of the diffractive optical element according to the experimental example. In addition, after preparing base compositions having different contents of nanoparticles (TiO₂) and photocurable polymers (Acrylate), transmittance of a coating layer formed from each of the base compositions is measured. More particularly, transmittance is measured for a coating layer formed from each of the base compositions having a content of nanoparticles (TiO₂):photocurable polymer (Acrylate) in the ratios of 9:1, 8:2, and 7:3.

As shown in FIG. 14, all of the adhesive layer (PMMA) and the coating layers formed of the base compositions having contents of 9:1 and 7:3 have high transmittance of 80% or more within the visible light range (400 nm to 800 nm). In addition, it can also be confirmed that the coating layer formed of the base composition having the content of 8:2 has high transmittance approaching 80%.

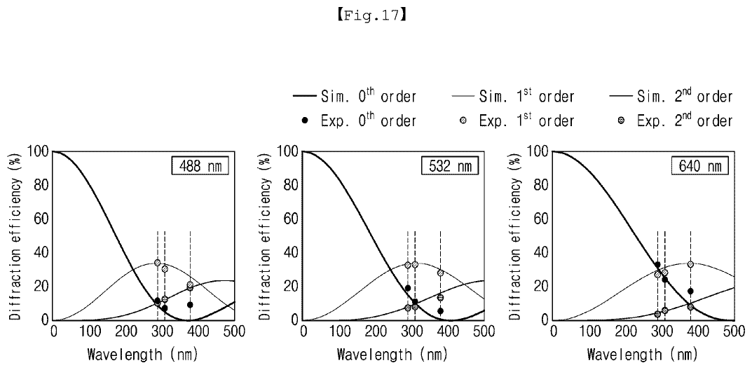

FIG. 15 is a view for explaining results of measuring refractive index of a coating layer in the diffractive optical element according to the experimental example of the present invention.

Referring to FIG. 15, the refractive index is measured for each of the azopolymer film (Azopolymer) and the coating layer (High refractive index resin) formed from the base composition having the content of nanoparticles (TiO₂):photocurable polymer (Acrylate) in the ratio of 9:1.

As shown in FIG. 15, the coating layer in the diffractive optical element according to the experimental example has a higher refractive index compared to the azopolymer film. As a result, as shown in FIGS. 14 and 15, the diffractive optical element according to the experimental example is transparent and has a high refractive index.

### Experimental Example 3: Diffraction Efficiency and Diffraction Tendency of Diffractive Optical Element

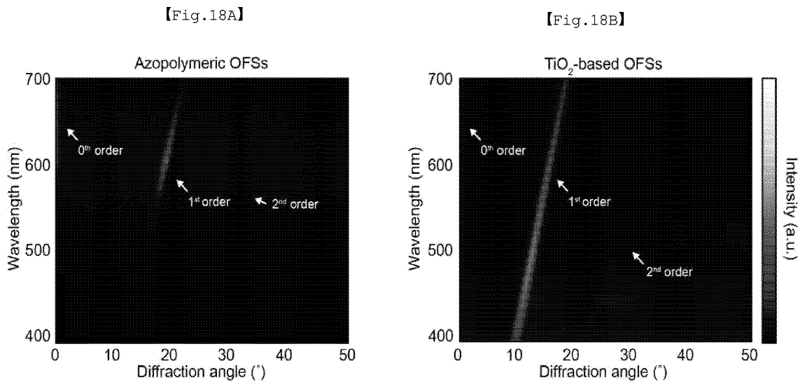

FIG. 16 is a view for explaining a theoretical diffraction efficiency of the diffractive optical element according to the experimental example of the present invention.

Referring to FIG. 16, the theoretical diffraction efficiency of the diffractive optical element according to the experimental example is derived using <Equation 2> below, and the derived result values are shown.

$$\\ \begin{matrix}{DE}_{m} = {\vert \sum {J}_{m} \frac{{k}_{o} ( {n}^{′} - n ) {h}_{0}}{2} \vert}^{2} & < Equation 2 >\end{matrix}$$

DEₘ: diffraction efficiency, m: diffraction order, k₀: frequency, n′: refractive index of diffractive optical layer, n: refractive index of air (=1), h₀: maximum value of modulation height, J: Bessel function)

As can be seen in FIG. 16 and <Equation 2>, the refractive index of the diffractive optical layer (coating layer) and the maximum value of the modulation height are major variables for determining the diffraction efficiency. In addition, in the case that the diffraction efficiency is compared by setting n_{low} to 1.7 and n_{high} to 2.0 for the refractive index values, it can be seen that a maximum diffraction efficiency may be achieved at a lower modulation height when the refractive index values are higher.

Accordingly, it can be seen that theoretically maximum diffraction efficiency can be achieved because the refractive index can be further improved when the sinusoidal wave-shaped pattern is further formed on the coating layer having the high transmittance and the high refractive index characteristics according to the present invention,

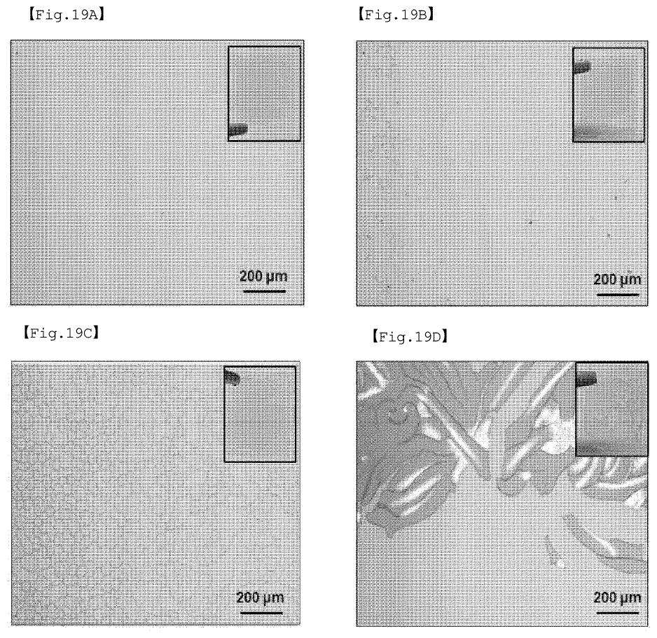

FIG. 17 is a view comparing an actual diffraction efficiency with a theoretical diffraction efficiency of the diffractive optical element according to the experimental example of the present invention.

FIG. 17 shows the comparison between the actual diffraction efficiency (Exp.) and the theoretical diffraction efficiency (Sim.) of the diffractive optical element according to the experimental example. More particularly, FIG. 17(a) shows results obtained by measuring light having a wavelength of 488 nm, FIG. 17(b) shows results obtained by measuring light having a wavelength of 532 nm, and FIG. 17(c) shows results obtained by measuring light having a wavelength of 640 nm. In addition, A, B and C shown in (a) to (c) of FIG. 17 denote diffractive optical elements having different modulation heights.

As shown in FIG. 17, all of the diffractive optical elements having different modulation heights have actual diffraction efficiencies close to the theoretical maximum diffraction efficiency for the light having wavelengths from 488 nm to 640 nm.

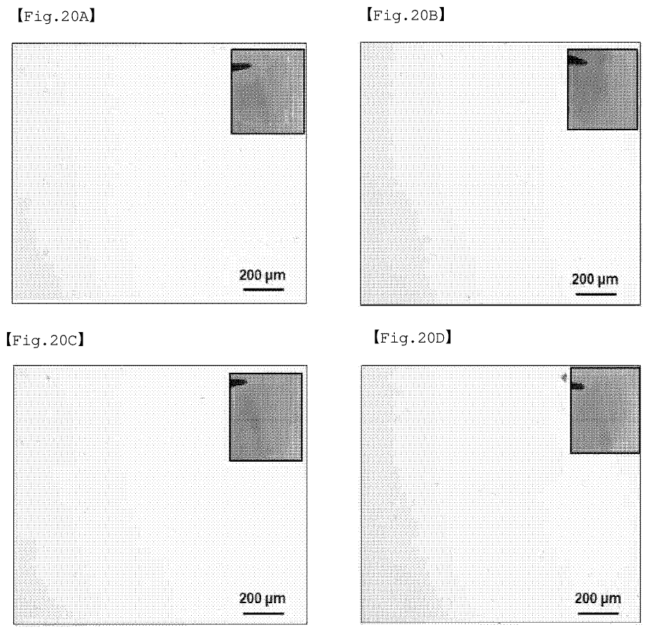

FIGS. 18A and 18B are views comparing diffraction tendencies between the diffractive optical element according to the experimental example of the present invention and a diffractive optical element according to a comparative example.

Referring to FIG. 18A, a diffractive optical element having a sinusoidal wave-shape polymer pattern formed by irradiating light containing a polarization interference pattern on a polymer film containing azobenzene molecules is prepared as a diffractive optical element according to a comparative example, and then a diffraction tendency for the prepared diffractive optical element is measured and indicated. Referring to FIG. 18B, the diffractive optical element according to the experimental example is prepared and then a diffraction tendency for the prepared diffractive optical element is measured and indicated.

As shown in FIGS. 18A and 18B, a diffraction signal for light having a wavelength of about 550 nm or less is not observed from the diffractive optical element according to the comparative example due to absorption of azobenzene molecules. However, a diffraction signal is clearly observed over the entire visible light range in the diffractive optical element according to the experimental example.

### Experimental Example 4: Determining Influences of Adhesive Work Values

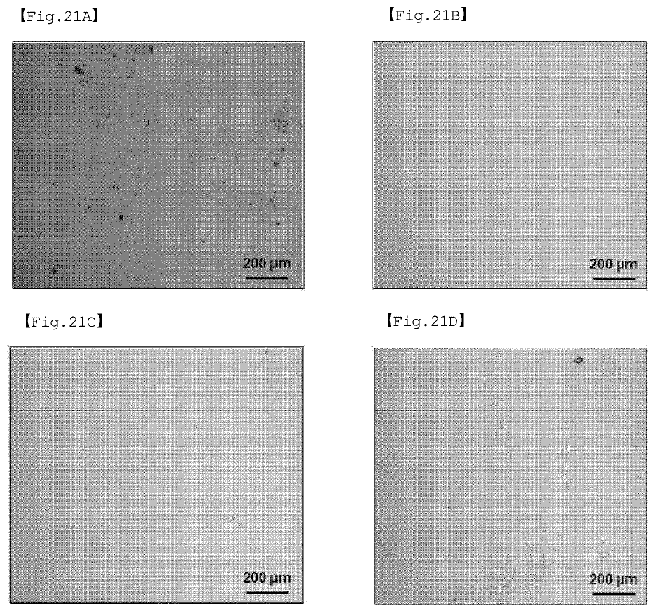

FIG. 19A to FIG. 20D are views for explaining influences of bonding work values in a manufacturing process of the diffractive optical element according to the experimental example of the present invention.

Referring to FIGS. 19A to 19D, four diffractive optical elements manufactured according to the above-described experimental example but manufactured under different conditions are prepared, and then states of each coating layer are photographed and indicated. The four diffractive optical elements manufactured under the different conditions are defined as samples 1-1 to 1-4, and FIGS. 19A to 19D show states of the coating layers of samples 1-1 to 1-4, respectively.

More particularly, sample 1-1 is a diffractive optical element manufactured under conditions in which both of the adhesive layer (PMMA) and the hydrophobicity-imparted layer (HMDS) are used; sample 1-2 is a diffractive optical element manufactured under conditions in which the adhesive layer (PMMA) is used but the hydrophobicity-imparted layer (HMDS) is not used; sample 1-3 is a diffractive optical element manufactured under conditions in which the adhesive layer (PMMA) is not used but the hydrophobicity-imparted layer (HDMS) is used; and sample 1-4 is a diffractive optical element manufactured under conditions in which neither the adhesive layer (PMMA) nor the hydrophobicity-imparted layer (HDMS) is used.

In addition, samples 1-1 to 1-4 all use benzyl methacrylate (BzMA) as a photocurable polymer, and a ΔW value is measured for each sample using <Equation 1> below. The manufacturing conditions and ΔW values of samples 1-1 to 1-4 are summarized in <Table 1> below.

$$\\ \begin{matrix}△ W = \vert 4 ( \frac{{γ}_{s 1}^{d} {γ}_{s 2}^{d}}{{γ}_{s 1}^{d} + {γ}_{s 2}^{d}} + \frac{{γ}_{s 1}^{p} {γ}_{s 2}^{p}}{{γ}_{s 1}^{p} + {γ}_{s 2}^{p}} ) - 4 ( \frac{{γ}_{s 2}^{d} {γ}_{s 3}^{d}}{{γ}_{s 2}^{d} + {γ}_{s 3}^{d}} + \frac{{γ}_{s 2}^{p} {γ}_{s 3}^{p}}{{γ}_{s 2}^{p} + {γ}_{s 3}^{p}} ) \vert & < Equation 1 >\end{matrix}$$

(γ^{d}: dispersion surface energy, γ^{p}: polar surface energy, s₁ the base substrate, s₂: the coating layer, and s₃: the imprinting stamp)

TABLE 1

| | | Adhesive | Hydrophobicity- | |
|---|---|---|---|---|
| | Photocurable | layer | imparted layer | ΔW |
| Item | polymer | (PMMA) | (HDMS) | (mJ/cm²) |
| Sample 1-1 | BzMA | O | O | 40.624 |
| Sample 1-2 | BzMA | O | X | 38.0347 |
| Sample 1-3 | BzMA | X | O | 30.5079 |
| Sample 1-4 | BzMA | X | X | 27.9186 |

As can be seen in FIGS. 19A to 19D, sample 1-1 having a ΔW value of 40 or more does not have defects formed on the surface of the coating layer. In contrast, samples 1-2 to 1-4 having a ΔW value of less than 40 have defects formed on the surface of the coating layer.

Referring to FIGS. 20A to 20D, four diffractive optical elements manufactured according to the above-described experimental example but manufactured under different conditions are prepared, and then states of each coating layer are photographed and indicated. The four diffractive optical elements manufactured under the different conditions are defined as samples 2-1 to 2-4, and FIGS. 20A to 20D show states of the coating layers of samples 2-1 to 2-4, respectively.

More particularly, sample 2-1 is a diffractive optical element manufactured under conditions in which both of the adhesive layer (PMMA) and the hydrophobicity-imparted layer (HMDS) are used; sample 2-2 is a diffractive optical element manufactured under conditions in which the adhesive layer (PMMA) is used but the hydrophobicity-imparted layer (HMDS) is not used; sample 2-3 is a diffractive optical element manufactured under conditions in which the adhesive layer (PMMA) is not used but the hydrophobicity-imparted layer (HDMS) is used; and sample 2-4 is a diffractive optical element manufactured under conditions in which neither the adhesive layer (PMMA) nor the hydrophobicity-imparted layer (HDMS) is used.

In addition, all of samples 2-1 to 2-4 use dipentaerythritol penta-/hexa-acrylate as a photocurable polymer, and a ΔW value is measured for each sample using <Equation 1> above. The manufacturing conditions and ΔW values of samples 2-1 to 2-4 are summarized in <Table 2> below.

TABLE 2

| | | Adhesive | Hydrophobicity- | |
|---|---|---|---|---|
| | Photocurable | layer | imparted layer | ΔW |
| Item | polymer | (PMMA) | (HDMS) | (mJ/cm²) |
| Sample | Dipentaerythritol | O | O | 48.9767 |
| 2-1 | penta-/hexa- | | | |
| | acrylate | | | |
| Sample | Dipentaerythritol | O | X | 46.3661 |
| 2-2 | penta-/hexa- | | | |
| | acrylate | | | |
| Sample | Dipentaerythritol | X | O | 46.0923 |
| 2-3 | penta-/hexa- | | | |
| | acrylate | | | |
| Sample | Dipentaerythritol | X | X | 43.4816 |
| 2-4 | penta-/hexa- | | | |
| | acrylate | | | |

As can be seen in FIGS. 20A to 20D, all of samples 2-1 to 2-4 do not have defects formed on the surface of the coating layer.

As a result, as can be seen from Experimental Example 4, the ΔW value is required to be 40 mJ/cm² or more in order to form a coating layer having high reliability without defects. In addition, it can be seen that when BzMA is used as a photocurable polymer, the adhesive layer (PMMA) and the hydrophobicity-imparted layer (HDMS) are required to have the ΔW value of 40 mJ/cm² or more. In contrast, it can be seen that when dipentaerythritol penta-/hexa-acrylate is used as a photocurable polymer, the ΔW value is 40 mJ/cm² or more regardless of the adhesive layer (PMMA) and the hydrophobicity-imparted layer (HDMS).

### Experimental Example 5: Determining Influence of Contents of Photocurable Polymer and Nanoparticles

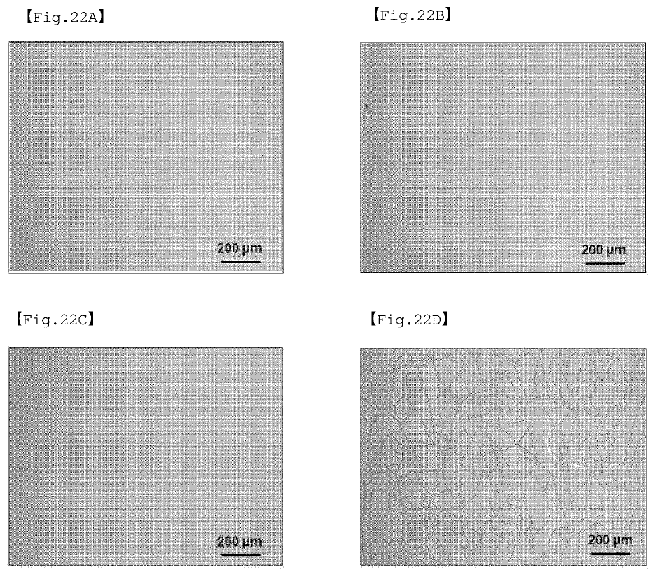

FIG. 21A to FIG. 22D are views for explaining influences of a photocurable polymer and a nanoparticle content in the manufacturing process of the diffractive optical element according to the experimental example of the present invention.

Referring to FIGS. 21A to 21D, four diffractive optical elements manufactured according to the above-described experimental example but manufactured under different conditions are prepared, and then states of each coating layer are photographed and indicated. The four diffractive optical elements manufactured under the different conditions are defined as samples 3-1 to 3-4, and FIGS. 21A to 21D show states of the coating layers of samples 3-1 to 3-4, respectively.

More particularly, samples 3-1 to 3-4 are manufactured under different conditions with different contents of photocurable polymer and nanoparticles in the base composition to form the coating layer, and dipentaerythritol penta-/hexa-acrylate is used as a photocurable polymer.

In addition, sample 3-1 is a diffractive optical element manufactured under conditions in which the content of nanoparticles: photocurable polymer is 70:30 wt %; sample 3-2 is a diffractive optical element manufactured under conditions in which the content of nanoparticles: photocurable polymer is 80:20 wt %; sample 3-3 is a diffractive optical element manufactured under conditions in which the content of nanoparticles: photocurable polymer is 90:10 wt %; and sample 3-4 is a diffractive optical element manufactured under conditions in which the content of nanoparticles: photocurable polymer is 95:5 wt %.

TABLE 3

| | | Nanoparticles: photo |
|---|---|---|
| | | curable polymer |
| Item | Photocurable polymer | (wt %) |
| Sample 3-1 | Dipentaerythritol penta-/hexa- | 70:30 |
| | acrylate | |
| Sample 3-2 | Dipentaerythritol penta-/hexa- | 80:20 |
| | acrylate | |
| Sample 3-3 | Dipentaerythritol penta-/hexa- | 90:10 |
| | acrylate | |
| Sample 3-4 | Dipentaerythritol penta-/hexa- | 95:5 |
| | acrylate | |

As can be seen in FIGS. 21B and 21C, the coating layers of samples 3-2 and 3-3 have no defects (aggregation or cracks). However, as can be seen in FIGS. 21A and 21D, aggregation occurs in the coating layer of sample 3-1, and cracks are formed in the coating layer of sample 3-4.

In other words, it can be seen that, when dipentaerythritol penta-/hexa-acrylate is used as the photocurable polymer for forming the coating layer in the process of manufacturing the diffractive optical element according to the embodiment of the present invention, the content of nanoparticles: photocurable polymer is required to be controlled to be greater than 70:30 wt % and less than 95:5 wt % in order to form the coating layer having high reliability.

Referring to FIGS. 22A to 22D, four diffractive optical elements manufactured according to the above-described experimental example but manufactured under different conditions are prepared, and then states of each coating layer are photographed and indicated. The four diffractive optical elements manufactured under the different conditions are defined as samples 4-1 to 4-4, and FIGS. 22A to 22D show states of the coating layers of samples 4-1 to 4-4, respectively.

More particularly, samples 4-1 to 4-4 are manufactured under different conditions with different contents of photocurable polymer and nanoparticles in the base composition to form the coating layer, and benzyl methacrylate (BzMA) is used as a photocurable polymer.

In addition, sample 4-1 is a diffractive optical element manufactured under conditions in which the content of nanoparticles: photocurable polymer is 70:30 wt %; sample 4-2 is a diffractive optical element manufactured under conditions in which the content of nanoparticles: photocurable polymer is 80:20 wt %; sample 4-3 is a diffractive optical element manufactured under conditions in which the content of nanoparticles: photocurable polymer is 90:10 wt %; and sample 4-4 is a diffractive optical element manufactured under conditions in which the content of nanoparticles: photocurable polymer is 95:5 wt %.

TABLE 4

| | Photocurable | Nanoparticles: photocurable |
|---|---|---|
| Item | polymer | polymer (wt %) |
| Sample 4-1 | BzMA | 70:30 |
| Sample 4-2 | BzMA | 80:20 |
| Sample 4-3 | BzMA | 90:10 |
| Sample 4-4 | BzMA | 95:5 |

As can be seen in FIGS. 22A to 22C, the coating layers of samples 4-1 to 4-3 have no defects (cracks). However, as can be seen in FIG. 22D, cracks are formed in the coating layer of sample 4-4.

In other words, it can be seen that, when benzyl methacrylate (BzMA) is used as the photocurable polymer for forming the coating layer in the process of manufacturing the diffractive optical element according to the embodiment of the present invention, the content of nanoparticles: photocurable polymer is required to be controlled to be less than 95:5 wt % in order to form the coating layer having high reliability.

Although the present invention has been described in detail with reference to the preferred embodiments, the present invention is not limited to the specific embodiments and will be interpreted by the following claims. Further, it will be apparent that a person having ordinary skill in the art may carry out various deformations and modifications for the embodiments described as above within the scope without departing from the present invention.

### INDUSTRIAL APPLICABILITY

The present invention may be used in the semiconductor industry.

1. A method for manufacturing a diffractive optical element, the method comprising:
preparing an imprinting stamp including a stamp pattern in which peaks and valleys having a curved shape are alternately and repeatedly arranged in at least some areas;
preparing a base structure in which a coating layer including a photocurable polymer and nanoparticles is formed on a base substrate; and
applying pressure to the coating layer of the base structure using the imprinting stamp to form, in the coating layer, a base pattern having the reverse phase of the stamp pattern.

2. The method of claim 1, wherein the preparing of the imprinting stamp includes:
preparing a sub-structure in which a polymer film containing azobenzene molecules is formed on a sub-substrate;
irradiating light onto the polymer film to form a polymer pattern in which peaks and valleys having a curved shape are alternately and repeatedly arranged in at least some areas;
coating a stamp composition including a thermosetting polymer on the polymer film to cover the polymer pattern; and
manufacturing the imprinting stamp including the stamp pattern having a reverse phase of the polymer pattern by heat-treating the stamp composition.

3. The method of claim 1, wherein the base pattern has peaks and valleys having a curved shape alternately and repeatedly arranged, in which the peak of the base pattern corresponds to the valley of the stamp pattern, and the valley of the base pattern corresponds to the peak of the stamp pattern.

4. The method of claim 1, wherein the preparing of the base structure includes:
preparing a base substrate;
forming an adhesive layer on the base substrate; and
forming the coating layer on the adhesive layer.

5. The method of claim 4, wherein the adhesive layer includes poly(methyl methacrylate) (PMMA).

6. The method of claim 4, further including:
treating the base substrate by using oxygen plasma, after the preparing of the base substrate and before the forming of the adhesive layer.

7. The method of claim 1, further comprising:
imparting hydrophobicity to a surface of the imprinting stamp on which the stamp pattern is formed, after the preparing of the imprinting stamp and before the forming of the base pattern.

8. The method of claim 7, wherein the surface of the imprinting stamp on which the stamp pattern is formed is treated with hexamethyldisilazane (HMDS) to impart the hydrophobicity.

9. The method of claim 1, wherein the surface of the imprinting stamp on which the stamp pattern is formed is further disposed thereon with a hydrophobicity-imparted layer, and
the base structure further includes an adhesive layer disposed between the base substrate and the coating layer, such that a ΔW value derived through <Equation 1> below is 40 mJ/m² or more
$$\\ \begin{matrix}△ W = \vert 4 ( \frac{{γ}_{s 1}^{d} {γ}_{s 2}^{d}}{{γ}_{s 1}^{d} + {γ}_{s 2}^{d}} + \frac{{γ}_{s 1}^{p} {γ}_{s 2}^{p}}{{γ}_{s 1}^{p} + {γ}_{s 2}^{p}} ) - 4 ( \frac{{γ}_{s 2}^{d} {γ}_{s 3}^{d}}{{γ}_{s 2}^{d} + {γ}_{s 3}^{d}} + \frac{{γ}_{s 2}^{p} {γ}_{s 3}^{p}}{{γ}_{s 2}^{p} + {γ}_{s 3}^{p}} ) \vert & < Equation 1 >\end{matrix}$$
(γ^{d}: dispersion surface energy, γ^{p}: polar surface energy, s₁: the base substrate, s₂: the coating layer, and s₃: the imprinting stamp).

10. A diffractive optical element comprising:
a diffractive optical layer including a polymer and nanoparticles, wherein
the diffractive optical layer includes a base pattern in which peaks and valleys having a curved shape are alternately and repeatedly arranged, and has a diffraction efficiency of 30% or more.

11. The diffractive optical element of claim 10, wherein the diffractive optical element has transmittance of 80% or more for light having a wavelength of 400 nm to 800 nm.

12. The diffractive optical element of claim 10, wherein the polymer includes dipentaerythritol penta-/hexa-acrylate or benzyl methacrylate (BzMA).

13. The diffractive optical element of claim 10, wherein the nanoparticles include titanium oxide (TiO₂) nanoparticles.
